(19) United States
(12) Patent Application Publication
(10) Pub. No.: US 2010/0151517 A1
(43) Pub. Date: Jun. 17, 2010

(54) METHODS FOR CARRYING OUT THE SELECTIVE EVOLUTION OF PROTEINS IN VIVO

(75) Inventor: Michael Liss, Regensburg (DE)

Correspondence Address: ROTHWELL, FIGG, ERNST & MANBECK, P.C., 1425 K STREET, N.W., SUITE 800, WASHINGTON, DC, 20005, US

(73) Assignee: Geneart AG, Regensburg (DE)

(21) Appl. No.: 11/917,957

(22) PCT Filed: Aug. 7, 2006
(86) PCT No.: PCT/EP06/07797

§ 371 Date: Dec. 18, 2007

(30) Foreign Application Priority Data
Aug. 8, 2005 (DE) 10 2005 037 349.6

(51) Int. Cl. C12P 21/00; C12N 5/00; C12N 1/00

(52) U.S. Cl. 435/69.1; 435/325; 435/243

(57) ABSTRACT

The present invention relates to methods for producing variants of proteins which have improved properties in comparison with the initial protein, the variants being obtained with the aid of an in vivo evolution method.

The present invention relates to methods for producing variants of proteins which have improved properties by comparison with the initial protein, the variants being obtained with the aid of an in vivo evolution method.

### BACKGROUND OF THE INVENTION

The increasing importance of biotechnology in the medical, chemical industry and agronomic sectors means that there is an increasing demand for proteins optimally adapted for their particular purpose of use. These proteins are initially isolated mainly from the environment, mostly within the framework of so-called metagenomic screenings. Increasingly, they are subsequently adapted by various methods to the planned “artificial” use conditions.

Thus, for example, there are needs for enzymes which are more thermally stable than their natural variants, have a different substrate specificity or show higher activities. Pharmaceutical proteins for instance are intended to have longer half-lives in order to be able to use smaller doses, or to inhibit, via the high-affinity and specific binding to target molecules, disease-associated metabolic pathways or infection routes.

### PRIOR ART

This adaptation takes place in part by rational protein design approaches. However, the present possibilities for concluding the structure of a protein from its desired function—the phenotype—and for inferring the corresponding primary sequence from the three-dimensional structure of the protein are only very limited. In order nevertheless to achieve an increase in the function of a protein, the approaches used at present are partly evolutionary and are summarized by the term “directed evolution” (Buskirk et al., 2003 “In vivo evolution of an RNA-based transcriptional activator”. Chem. Biol. 10:533-540; de Crécy-Lagard et al., 2001, “Long term adaptation of a microbial population to a permanent metabolic constraint: overcoming thymineless death by experimental evolution of Escherichia coli”, BMC Biotechnology 1:10; Fields and Song, 1989, “A novel genetic system to detect protein-protein interactions”, Nature, 340: 245-246; Long-McGie et al., 1999, “Rapid in vivo evolution of a β-lactamase using phagemids”, Biotechnol. Bioeng. 68:121-125; Rohde et al., 1995, “The mutant distribution of an RNA species replicated by Q beta replicase”, J. Mol. Biol. 249:754-762).

Methods of directed evolution to date are based, according to the current prior art, substantially on generating a large number of variants (progeny) of the protein to be improved, and selection thereof for improved derivatives. In this case, the number of investigated mutants may in some cases be very large, but is usually below 10¹¹. Considering a protein of only 100 amino acids, in theory 20¹⁰⁰=10¹³⁰ different variants thereof exist. A library with a size of 10¹¹ accordingly covers only a very small fraction of the possible variants. The probability of finding the theoretically best variant in such a library is approximately zero.

The principle of evolution with its three preconditions—replication, mutation and selection—is capable within a given system of bringing about directed evolution from simple to highly adapted structures. This model of the so-called “blind watchmaker” enables complexity to be created without a design input and without necessary knowledge of structural data.

In order to screen a large number of variants for maximum affinity for a target molecule, a large number of protocols has already been developed (e.g. yeast two-hybrid, bacterial display, phage display, ribosomal display, mRNA display). Screening for other properties such as, for instance, enzymatic activity mostly requires assay formats which permit the investigation of only a relatively small number of variants (<10⁶).

It is common to these methods that they are confined to the generation of a library (=mutation) and subsequent selection thereof. Although a replication (=next generation of mutants of the “winner” of the selection which took place last) is possible manually with these protocols, it is just as complicated as the preceding step. Corresponding approaches therefore generally extend only over one to two generations. For this reason, the protocols mentioned are not evolutionary approaches in the true sense but, on the contrary, are exclusive selection of available pools of variants.

Consequently, the potential for stepwise adaptation over many generations cannot be utilized, but this would be the necessary precondition for identifying in some circumstances the most active variant from an astronomically large number of possibilities.

WO 2004/108926 discloses methods for evolving nucleic acids and proteins. This method is based on the utilization of the high mutation rate and adaptability of RNA viruses, especially the bacteriophage Phi. A β-lactamase gene was inserted into the genome of the bacteriophage Phi6 to form a recombinant phage. This recombinant Phi6 phage was propagated in bacterial cells with avoidance of lysis, a selection pressure being exerted by adding ampicillin and a further antibiotic. It was found that the surviving bacterial cells contained phage RNA molecules which contained a β-lactamase gene modified at the nucleic acid level compared with the originally introduced β-lactamase gene. The selection pressure had thus led to evolution of the β-lactamase gene. Mention is likewise made of further possibilities for evolution of, for example, regulatory proteins or RNAs or molecules having specific binding properties. However, the possibility of evolution of any desired proteins which are completely independent in terms of their function from the expression of the respective section gene is not disclosed. In addition, WO 2004/108926 envisages a separate selection/screening step. The disclosure concerning the evolution of molecules having particular binding properties in particular requires a screening after each generation of evolution, so that the disclosed system cannot be left completely to itself.

The object of the present invention was therefore to create a system which provides an in vivo evolution method which does not depend on elaborate screening methods.

This object is achieved by a method for producing variants Y′ of a protein Y, where the variants Y′ of the protein Y are characterized by modified binding properties to a target molecule X, comprising the steps:

(a) provision of a cell comprising
(a1) a first nucleic acid which codes for a protein Y to be varied,
(a2) a second nucleic acid which codes for the target molecule X, and
(a3) a third nucleic acid which codes for an evolution marker under the control of an expression control sequence which can be modulated,
   where the expression of the evolution marker is modulated by the binding of Y and/or Y′ to X,
      (b) cultivation of the cell under conditions which
(b1) enable formation of variants Y′ of the protein Y and
(b2) permit selection for those cells which exhibit an expression, modulated by comparison with the cell from (a), of the evolution marker, and
   (c) identification and, where appropriate, isolation of those cells which exhibit a modulated expression of the evolution marker, and
   (d) where appropriate identification and/or characterization of variants Y′ of the protein Y in the cells from (c).

This invention encompasses autonomous systems which on the one hand permit selection of a given variant library in the laboratory, and simultaneously include a replication mechanism. The selection in this case is to be implemented solely—as in natural evolution—via a preferred replication of better-adapted variants. In addition, the replication system used is designed so that it permits an adequate mutation rate during the replications. The number of variants, theoretically tested in this way, of an initial construct is calculated from the number of progeny of a winner of one generation to the power of the total number of generations in an experiment (e.g. 10 progeny per generation with 400 generations=10⁴⁰⁰ possible variants.) Although it is impossible for each of these 10⁴⁰⁰ individual variants explicitly to be present physically in one experiment, the system itself looks for a “path”, within a complex virtual terrain, which always leads upwards to the absolute maximum. Only the variants along the path have existed during the experiment; all points (variants) of the terrain are theoretically possible.

The method of the invention is thus based on the principle that a protein Y which is to be evolved and which, for the purposes of the present invention, is referred to as “protein Y to be varied” is encoded by a nucleic acid which leads on expression in vivo to variants Y′ of this protein, the formation of variants taking place at the nucleic acid level, e.g. through replication of the nucleic acid which codes for Y, through polymerases with a particular error rate (e.g. because they have no proofreading function).

These variants Y′ can then be selected for various properties such as, for example, for the property of binding to a particular further protein with higher affinity. If these higher-affinity binding properties of the variants Y′ of the protein Y are suitable for activation and/or enhancement of expression of an evolution marker gene, on application of selection pressure it is possible for evolution to take place autonomously, because the only cells to survive or propagate or propagate more quickly are those which express variants of the protein Y having the desired improved properties.

In principle, any type of cells are suitable for a system of this type, possibilities being prokaryotic cells or eukaryotic cells. The type of cell depends where appropriate on the selection system to be chosen, the skilled person being able to select cells suitable for the particular selection system. It is possible to use bacterial cells, yeast cells, plant cells and vertebrate cells, especially mammalian cells, preferably human cells. The selection of the cell depends on the particular evolution system and can be selected by the skilled person himself.

As already mentioned above, the method of the invention includes two groups of processes, namely on the one hand replication and mutation and on the other hand selection. Methods and systems for both these groups of processes have been disclosed in the prior art.

In order for a protein Y to be varied to evolve to variants Y′ having improved properties, it is initially necessary to provide the possibility of development of such variants in an in vivo environment, i.e. for example in a cell. This can take place for example at the nucleic acid level. For example, it is possible to employ DNA polymerases or RNA polymerases which exhibit a certain error rate, so that mutated nucleic acids arise during replication and/or transcription of the nucleic acid which codes for the protein Y and then lead, e.g. owing to point mutations, deletions or insertions, at the protein level to variants Y′ of the protein Y to be varied.

The polymerases responsible for the replication and mutation, i.e. DNA-dependent and RNA-dependent DNA polymerases and RNA polymerases, may, depending on the chosen embodiment of the method of the invention, either already be present in the cell, or they can also be provided separately, for example they can be encoded by a further nucleic acid, e.g. a plasmid.

The first nucleic acid which codes for the protein Y to be varied is according to the invention in a form which can be replicated at the nucleic acid level by polymerases in the cell. In a preferred embodiment, the first nucleic acid is in the form of an RNA replicon.

For the purposes of the present invention, “replicon” means in particular a nucleic acid which is replicated by an RNA-dependent RNA polymerase, specifically a so-called replicase. A replicon can thus either be an RNA, or else a replicon can be DNA which codes after a first transcription cycle for an RNA replicon. The RNA replicon is preferably selected from linear RNA sequences, genomes from RNA-based organisms, RNA plasmids, RNA viruses such as, for example, RNA bacteriophages, and RNA analogs. The replicases suitable according to the invention are preferably selected from RNA-dependent RNA polymerases such as, for instance, Qβ, Phi6, Phi8, Phi9, Phi10, Phi11, Phi13 and Phi14 replicases, and the like (see, for example, WO 2004/108926 and the publications by Makajev et al. mentioned below).

Replicases are far less accurate than DNA polymerases because they have no proofreading function. Their error rate is one mutation per 10³ to 10⁴ synthesized bases. In addition to the high error rate, however, replicases, especially Qβ replicase, are highly substrate-specific and can thus be “calibrated” for specific target RNAs. Replicases known in the state of the art are, for example, Qβ replicase or Phi replicases. Phi replicases and their evolutionary potential have been described in the prior art, especially by Makajev et al. in Journal of Virology, 2004, volume 78, No. 4, pages 2114-2120, EMBO Journal, 2000, volume 19, No. 1, pages 124-133 and Virus Research, 2004, 101, 45-55 and in the international patent application WO 2004/108926. Said patent application describes a system in which a modified genome of the bacteriophage Phi6 is used as replicon, and the viral Phi6 replicase is used as replicase. The system described therein is carried out in E. coli cells. A system of this type is also suitable for the present invention.

The second nucleic acid codes for a target molecule. The target molecule X is preferably a protein, but may also be another gene product obtained by expression of the second nucleic acid, such as, for instance, an mRNA or nucleic acid derivatives thereof. The target molecule X is chosen for the purposes of the present invention in such a way that the protein Y and its variants Y′ bind to this target molecule X, and binding of X and Y or X and Y′ can modulate the expression of the evolution marker. The nucleic acid which codes for the target molecule X can be provided in any suitable form, e.g. in the form of a plasmid which is suitable for transfection or transformation of cells. This plasmid is thus preferably equipped with a selection marker. The target molecule X can for the purposes of the present invention be chosen completely unrestrictedly, and it is thus possible to select any target molecule which acts as binding partner for the protein Y to be varied which is sought.

In order to carry out the directed evolution according to the invention it is necessary to exert a certain selection pressure on the selected cell system. For this reason, the third nucleic acid codes for an evolution marker under the control of an expression control sequence which can be modulated, with expression of the evolution marker being modulated by binding of the protein Y or its variants Y′ to a target molecule X. The third nucleic acid is preferably provided in the form of a plasmid, but can be in any form suitable for introduction into a cell. The expression control sequence which can be modulated preferably includes at least one promoter and particularly preferably further sequences, e.g. enhancers and the like, especially upstream activating sequences such as, for example, activating sequences on the DNA which permit the binding of transcription modulators, especially the binding of transcription activators, where the binding of these transcription modulators has an influence on the expression of the corresponding gene which is controlled by the expression control sequence which can be modulated.

The evolution marker is for the purposes of the invention a gene which codes for a gene product which makes it possible to select those cells which express the evolution marker. In prokaryotic cells, in principle every antibiotic resistance gene is suitable. Some examples are, for example, ampicillin resistance genes such as, for instance, the bla gene which codes for β-lactamase. Further such resistance genes are those which code for example for aminoglycoside 3′-phosphotransferase (nptII), which mediate resistance to kanamycin and G418, and chloramphenicol acetyltransferase (cat). The evolution marker preferred for a prokaryotic system is cat.

Evolution markers which can be used in yeast systems are the following genes, e.g. his3, trp1, ura3 or leu2. Suitable in this connection in prokaryotic and yeast systems is for example the selection gene his3 which mediates histidine prototrophy and whose dose effect can be adjusted via the concentration of 3-amino-triazole in the medium. Similar resistance genes can be used as evolution markers in other cases too, such as, preferably, mammalian cells.

Expression of the evolution marker permits selection for example in such a way that the only cells to be selected are those which propagate more rapidly than those cells in which the evolution marker is expressed less well or not at all (where the evolution marker is a resistance gene), or cells which express the expression marker less well or not at all (if the evolution marker is a gene which is disadvantageous for the cell).

Thus, the growth rate of a cell is directly related to the expression of the selection gene.

A further possibility is also to use conventional selection markers as evolution markers. These evolution markers may for example code for antibiotic resistance genes or the like.

A further modification of the selection system consists of using as evolution marker a gene which codes for a protein which is expressed on the cell surface. It is then possible for cells which express this protein, owing to the binding of the expressed protein to a corresponding binding partner which is coupled to a solid matrix, to be bound to this solid matrix. This then makes it possible to select the cells expressing the evolution marker from other cells which are not able to bind to the solid matrix.

The evolution marker is under the control of an expression control sequence which can be modulated. Suitable for this purpose are all types of promoters, enhancers and similar activator sequences which can be activated in trans and/or in cis. In a preferred embodiment, the expression control sequence is a sequence which includes a sequence which requires the binding of a protein for activation. One example is an upstream activating sequence (UAS).

The principle of the two-hybrid system is preferably used for such a modulation of the evolution marker. This system is well known in the state of the art and is used to detect protein-protein interactions. The two-hybrid system is based on a complex of two fusion proteins, where one fusion protein has a binding domain for the activating sequence (UAS) located in the expression control sequence, fused to or coupled to a protein X. The second protein complex comprises a protein Y to be investigated and an activator domain which interacts with the polymerase responsible for transcription. Only when binding takes place between X and Y can the expression control sequence be activated and the gene controlled thereby, i.e. the selection gene, be expressed.

It is clear to a skilled person that this system of two binding partners X and Y or Y′ can be used very well for the method of the present invention. A great disadvantage of the yeast two-hybrid system is, however, that it is suitable only for detecting protein-protein interactions between two known proteins or for screening previously generated protein variants. The two-hybrid system known in the state of the art exhibits no protein evolution step.

The method of the invention by contrast permits the generation of variants Y′ of the protein Y in the provided system, i.e. within the cell, automatically. It is thus possible to allow development of the proteins Y to be varied of their own accord in a particular direction which can be controlled by the binding ability of the variants Y′ to the target molecule X.

If there are generated in the system variants of Y which have a higher affinity for X, or in contrast to Y in fact have an affinity for X, expression of the selection gene allows survival and faster propagation and thus selection of those cells having a variant Y′ of the varying protein with improved properties.

In the well-known yeast two-hybrid system, the expression control sequence is activated by using the protein Gal4 which has a binding domain for an upstream activating sequence, and an activating domain which interacts with the polymerase. It is possible to separate these two domains of the Gal4 protein and provide each domain in each case with a further molecule either as fusion protein or to couple in another way, in which case the interaction between the further molecules, e.g. X and Y or Y′, makes it possible to bring the two Gal4 domains into spatial proximity with one another again, enabling modulation of the expression control sequence.

The second and third nucleic acid may be present either as separate nucleic acids, e.g. in the form of plasmids, or they may also both be present on the same plasmid. The first nucleic acid should, as already mentioned above, be a replicable nucleic acid, preferably an RNA replicon which can be replicated by RNA replicases. However, it is also possible to provide the first nucleic acid in the form of a plasmid or of another form of a transfectable or transformable nucleic acid, in which case this nucleic acid ought to code for a correspondingly replicable first nucleic acid.

It is possible for the purposes of the present invention to use the well-known yeast two-hybrid system or an equivalent system, where the interaction, i.e. the binding of the protein Y and/or variants Y′ of the protein Y to a target molecule, makes it possible to modulate the expression control sequence which can be modulated, and thus to modulate expression of the evolution marker.

Thus, there is preferably use of two expression modulators, where the interaction of the two expression modulators with one another, either directly or indirectly, via X and Y or Y′ is necessary for modulating the expression control sequence of the evolution marker.

In a preferred embodiment, the protein Y to be varied, which is encoded by the first nucleic acid, in particular an RNA replicon, is coupled to either the first or the second expression modulator. This involves either a fusion protein between Y and the first expression modulator or a fusion protein between Y and the second expression modulator or the protein Y is coupled in another way, e.g. via biotin-streptavidin binding, to one of the two expression modulators.

The target molecule X may be a protein or else another molecule, e.g. a nucleic acid. It is important that X either is coupled to an expression modulator or forms with the latter a fusion protein or itself represents the appropriate expression modulator.

In one embodiment, for example the first (or alternatively the second) expression modulator is a molecule which binds to an upstream activating sequence (UAS). The second (or alternatively the first) expression modulator modulates the expression through binding to the corresponding polymerase, with modulation of expression taking place only if there is simultaneously interaction of the first (or alternatively of the second) expression activator with the UAS and interaction of the second (or alternatively of the first) expression activator with the polymerase. Coupled or fused to this expression modulator are in each case the molecules X and Y or Y′, with the interaction between X and Y or between X and Y′ thus making it possible for the two expression modulators to be able to modulate the expression.

It is preferred in the present invention to activate the expression control sequence which can be modulated, whereby the evolution marker is expressed and thus the cell is provided with a growth advantage. However, it is also possible to use a system in which the modulation is an inhibition.

In a preferred embodiment which makes use of the two-hybrid system, the protein X and the protein Y are provided in each case as fusion proteins with the DNA binding domain (preferably as X/binding domain) and with the activator domain (preferably Y/activator domain). However, it is also possible instead of fusion proteins for the desired units to be coupled together via further molecules, for example via linkers or streptavidin/biotin bindings or the like.

Instead of the conventional two-hybrid system, it is also possible to use tri-hybrid systems, or protein complexes with more subunits can be used.

It is furthermore possible by the one-hybrid system to evolve proteins Y which bind to a given DNA sequence.

The experimental procedure in a preferred embodiment of the invention takes place initially via transformation of the first nucleic acid into a yeast or E. coli strain which already comprises the two other nucleic acids. The transformation mixture is then grown under evolution marker selection conditions in liquid culture, continuously monitoring the growth rate of the culture. Before the culture enters the logarithmic phase, an aliquot thereof is transferred into fresh medium, and further growth is observed. The transfer can also take place automatically, or via a continuous addition of medium and removal of culture.

An endpoint of the evolution process is reached when no further increase in the growth rate is observed despite the selection pressure being raised. Isolation and sequencing of the replicon sequence(s) of single colonies identifies potential candidates which are subsequently investigated individually for the affinities of their gene products. It is likewise possible to treat the totality of all variants present within the system at the endpoint as gene library, and to identify the variants which are best in turn via a selection assay (e.g. phage display).

FIG. 1 shows a diagrammatic representation of a preferred embodiment of the system of autonomous evolution in vivo, which is described in detail below.

The present invention further relates to a cell comprising
(i) a first nucleic acid which codes for a protein Y to be varied,
(ii) a second nucleic acid which codes for a target molecule X, where Y is able to bind to the target molecule X,
(iii) a third nucleic acid which codes for an evolution marker under the control of an expression control sequence which can modulated,
   and where the cell is able to permit the formation of variants Y′ of the protein Y,
   where the variants Y′ of the protein Y are characterized by modified binding properties to the target molecule X.

The cell can be transformed or transfected with the appropriate nucleic acids, it being possible to use conventional methods which are well known to the skilled person for the transformation and transfection.

The present invention further relates to a kit for carrying out the method of the invention, i.e. for developing variants Y′ of any protein Y, where the variants Y′ of the protein Y are characterized by modified binding properties to the target molecule X,
(i) where appropriate a cell, comprising
(ii) a first nucleic acid which codes for a protein Y to be varied,
(iii) a second nucleic acid which codes for a target molecule X, where Y is able to bind to the target molecule X,
(iv) a third nucleic acid which codes for an evolution marker under the control of an expression control sequence which can be modulated, and where the cell is able to permit the formation of variants Y′ of the protein Y, where the variants Y′ of the protein Y are characterized by modified binding properties to the target molecule X, and
(v) where appropriate a suitable selection medium.

The method disclosed herein has the advantage that it avoids elaborate screening methods to a large extent and that any proteins can be evolved.

It is possible in particular through the combination of directed evolution with a system which is based on the yeast two-hybrid system to achieve evolution of proteins having improved binding properties for a target molecule X autonomously. It is in this connection no longer necessary for the variants formed after each generation to be isolated and characterized; on the contrary, the system can be left to itself for a plurality of generations until the variants Y′ of the protein Y having the best binding properties for the appropriately selected target molecule X have formed automatically.

In order to make it possible by RNA replicon-based in vivo evolution also to improve extracellular proteins (e.g. immunoglobulins, Anticalins, etc.), receptors etc., it is possible for the target molecule X to be expressed in the cell in such a way that it is exported to the cell surface or into the cell periplasm and remains anchored in this compartment. The replicon-encoded interaction variants are likewise anchored on the cell surface and can where appropriate interact via a flexible linker domain with the target molecule X. An increased interaction in turn leads (possibly via a third molecule which is displaced by the interaction) to a selection advantage of the relevant cell. The signal can take place for example via a conformational change of the membrane-anchoring domain with subsequent signal cascade into the cell nucleus (e.g. optimizing the affinity of insulin for the insulin receptor).

It is also conceivable to regulate membrane transporters which convey either essential molecules into the interior of the cell or potential cytotoxins out of the cell.

It is also conceivable to express target molecule X as di-, tri- or multimeric protein, whereby, with increasing affinity between the molecules, there is crosslinking thereof and formation of clusters on the surface. This may have effects on the adhesion of the cell to surfaces, whereby in turn selection for strongly adherent cells is possible, whereas less strongly adhering cells are eliminated with increasing stringency.

Adhesion is likewise used to effect selection of replicon-encoded interaction variants Y′ which are expressed in cells on the cell surface in the following way. The target molecule X (protein, peptide, nucleic acid, other polymers, small organic or inorganic molecules (e.g. intermediate of an enzymatically catalyzed reaction)) is in this case bound to a solid or liquid matrix (or micelles) and brought into contact with the cell suspension. Cells which express higher-affinity variants have a greater affinity for the solid matrix. With increasing stringency (e.g. salt, pH, binding competitors etc.), weakly adherent cells are washed away, and the remaining cells are exposed to fresh medium, and new matrix is provided, while part of the old matrix is removed. This process can easily be automated.

Cells which express very good binders will not be detached again from the surface. Nevertheless, they are still capable where appropriate of cell division. Daughter cells, have, if the direct environment is occupied, the opportunity of reaching distant regions with fresh matrix. It is possible in this case to change the matrix type from step to step (where a “step” is defined as from the inoculation of new medium to the next inoculation with part of the matrix), for example magnetic beads and polyvinyl surfaces (Eliza plate). In this way, always only “newly occupied matrix” is transferred from one step to the next.

In each case (intra- and extracellularly) it is possible to increase the affinity not only for proteins but also for small organic or inorganic molecules. It is possible for this purpose to couple the molecule, covalently for example, to biotin. This molecule is introduced into the cytoplasm or into the extracellular medium (it may be membrane-penetrating). Instead of the target molecule X, in this case streptavidin (as fusion in the case of two-hybrid) is expressed. The target molecule/biotin chimera binds to streptavidin and thus presents the target molecule in order to act as affinity target.

### DESCRIPTION OF THE FIGURE

FIG. 1: Diagrammatic representation of the system for autonomous evolution in vivo

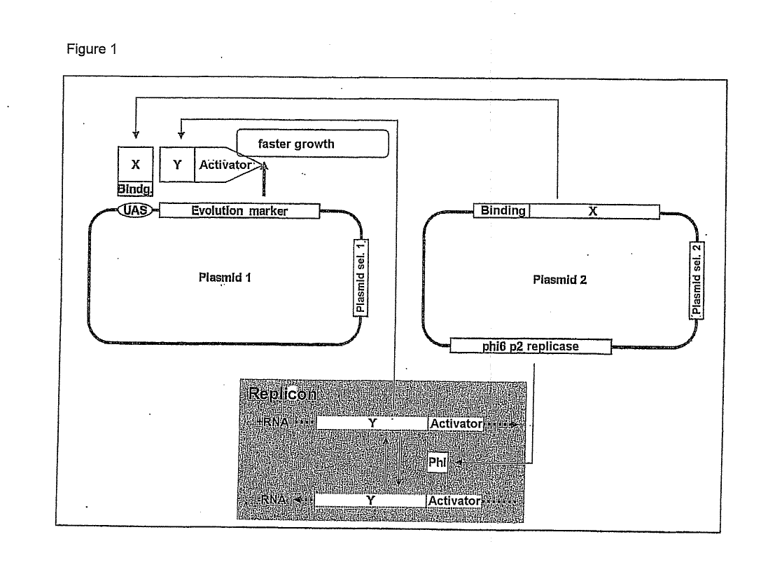

Plasmid 1 codes, under the control of the upstream activating sequence (UAS), for an evolution marker which mediates faster growth. The fusion protein composed of DNA binding domain and component X (binding/X) is encoded by plasmid 2 and binds to the UAS. Depending on the strength of the interaction between component X and the protein Y or Y′, the fused activating domain (activator) is able to recruit the cell's own transcription complex, and dependent expression of the evolution marker takes place. The fusion protein Y/activator is encoded by a replicon which undergoes autonomous and error-prone replication by an RNA-dependent RNA polymerase (Phi).

### Example

Starting from a synthetic gene, a PCR product comprising the following elements is generated: T7 promoter+reading frame for the activating domain of the yeast protein Gal4 (Fields and Sternglanz, 1994) fused to a peptide of 30 random amino acids (=Act/30X). Owing to the randomness of the 30 C-terminal amino acids, a gene library which comprised a number of approx. 10⁹different variants was generated. In a transcription reaction, 5 pmol of the PCR product were transcribed with T7 polymerase in a 100 μl reaction (=Act/30X replicon).

A yeast expression cassette for the RNA-dependent RNA polymerase Qβ (Acc. No. AAM33128) is cloned into the plasmid pAS (Fields and Sternglanz, 1994) (=pAS-Qβ). This plasmid permits selection for tryptophan prototrophy in yeast.

The reading frame for HIV integrase (Acc. No. AAC61700.1) is cloned, fused to the Gal DNA binding domain, into the plasmid pAS-Qβ (=pAS-Qβ-Int), transformed into the yeast strain Y190 and selected for tryptophan prototrophy.

The yeast culture transformed with pAS-Qβ-Int is transformed with 10 μg of Act/30X replicon RNA (transformation rate about 500 000) and directly incubated in 50 ml of tryptophan/histidine dropout liquid culture with addition of 12 mM 3-aminotriazole (3-AT) and with shaking at 30° C.

After an OD (600 nm) of 3.0 is reached, 50 ml of fresh medium are inoculated with 200 μl of the culture and again shaken until the OD reaches 3.0 (this corresponds to 8 generation doublings). The time to reach this OD divided by eight corresponds to the generation time of the yeast population.

If the generation time approaches 60 min, the 3-AT concentration is initially increased to 25 mM (after the 5th transfer) and then to 50 mM (after the 8th transfer). A generation time of 85 min is reached after the 10th transfer and falls no further even after a further transfer.

20 μl of the yeast culture of the 10th transfer culture are plated out on tryptophan/histidine dropout plates and incubated at 30° C. for 3 days. The largest colony is transferred into 30 ml of tryptophan/histidine dropout medium, grown to an OD of 3.0 and centrifuged. The cell pellet is disrupted with glass beads in STET buffer, phenol and chloroform, the aqueous supernatant is extracted 2× with chloroform, and nucleic acids present are precipitated with ethanol. The dried pellet (DNA & RNA) is resuspended in 100 μl of 10 mM Tris. 5 μl thereof are transcribed into DNA with reverse transcriptase with an oligonucleotide specific for the replicon, and amplified by PCR. The PCR product is subcloned into a suitable vector and transformed in E. coli.

Sequence analysis of 384 E. coli clones reveals a random distribution of different sequences which code for evolved Act/30X variants which have an increased affinity for HIV integrase. More recent investigations show repeating amino acid motifs within the variable peptide (30X) which are responsible for the increased affinity.

1. A method for producing variants Y′ of a protein Y,
where the variants Y′ of the protein Y are characterized by modified binding properties to a target molecule X, comprising the steps:
(a) provision of a cell comprising
(a1) a first nucleic acid which codes for a protein Y to be varied,
(a2) a second nucleic acid which codes for the target molecule X, and
(a3) a third nucleic acid which codes for an evolution marker under the control of an expression control sequence which can be modulated,
where the expression of the evolution marker is modulated by the binding of Y and/or Y′ to X,
(b) cultivation of the cell under conditions which
(b1) enable formation of variants Y′ of the protein Y and
(b2) permit selection for those cells which exhibit an expression, modulated by comparison with the cell from (a), of the evolution marker, and
(c) identification and, where appropriate, isolation of those cells which exhibit a modulated expression of the evolution marker, and
(d) where appropriate identification and/or characterization of variants Y′ of the protein Y in the cells from (c).

2. The method as claimed in claim 1, where the cell is a prokaryotic or eukaryotic cell.

3. The method as claimed in claim 2, where the cell is selected from E. coli, yeast cells, plant cells and vertebrate cells, especially mammalian cells.

4. The method as claimed in claim 1, where the first nucleic acid is replicated and/or transcribed with a variation rate which is increased by comparison with the natural variation rate.

5. The method as claimed in claim 1, where the first nucleic acid is in the form of an RNA replicon, and the cell from (a) additionally comprises a replicase able to replicate the RNA replicon to form variants.

6. The method as claimed in claim 5, where the RNA replicon is selected from linear RNA sequences, genomes from RNA-based organisms, RNA plasmids, RNA bacteriophages and RNA analogs.

7. The method as claimed in claim 5, where the replicase is selected from RNA-dependent RNA polymerases such as, for instance, 0, Phi6, Phi8, Phi9, PhilO, Phill, Phi13 and Phi14 replicases.

8. The method as claimed in claim 1, where the evolution marker is selected from genes whose expression makes faster propagation of the cell possible.

9. The method as claimed in claim 1, where the evolution marker is selected from genes which code for surface-associated proteins which make it possible for the cell to bind to a solid matrix.

10. The method as claimed in claim 1, where the method is carried out intracellularly.

11. The method as claimed in claim 1, where the protein Y and its variants Y′ are each coupled to a first expression modulator, and X is coupled to a second expression modulator, where an interaction of the first expression modulator and of the second expression modulator with the nucleic acid coding for the evolution marker is necessary for odulating the expression of the evolution marker.

12. The method as claimed in claim 1 where X is a protein.

13. The method as claimed in claim 11, where the first nucleic acid codes for a fusion protein composed of Y and the first expression modulator, and the second nucleic acid codes for a fusion protein composed of X and the second expression modulator, or where the first nucleic acid codes for a fusion protein composed of Y and the second expression modulator, and the second nucleic acid codes for a fusion protein composed of X and the first expression modulator.

14. The method as claimed in claim 11, where the first expression modulator is a modulator for a polymerase.

15. The method as claimed in claim 11, where the second expression modulator is a modulator for a polymerase.

16. The method as claimed in claim 14, where the expression control sequence which can be modulated on the third nucleic acid includes an upstream activating sequence (UAS), and the second expression modulator is a binding protein capable of specifically binding to the UAS.

17. The method as claimed in claim 15, where the expression control sequence which can be modulated on the third nucleic acid includes an upstream activating sequence (UAS), and the first expression modulator is a binding protein capable of specifically binding to the UAS.

18. The method as claimed in claim 1, where the expression control sequence which can be modulated includes an upstream activating sequence (UAS) which permits binding of the protein Ga14.

19. The method as claimed in claim 1, where the binding of Y and/or Y′ to X enables activation of the expression control sequence which can be modulated on the third nucleic acid.

20. A cell comprising
(i) a first nucleic acid which codes for a protein Y to be varied,
(ii) a second nucleic acid which codes for a target molecule X, where Y is able to bind to the target molecule X,
(iii) a third nucleic acid which codes for an evolution marker under the control of an expression control sequence which can modulated, and where the cell is able to permit the formation of variants Y′ of the protein Y, where the variants Y′ of the protein Y are characterized by modified binding properties to the target molecule X.

21. The cell as claimed in claim 20, where the first nucleic acid is in the form of an RNA replicon, and the cell additionally comprises a replicase able to replicate the RNA replicon to form variants.

22. The cell as claimed in claim 20, which is a prokaryotic or eukaryotic cell.

23. A kit for producing variants Y′ of a protein Y, where the variants Y′ of the protein Y are characterized by modified binding properties to the target molecule X, comprising
(i) where appropriate a cell,
(ii) a first nucleic acid which codes for a protein Y to be varied,
(iii) a second nucleic acid which codes for a target molecule X, where Y is able to bind to the target molecule X,
(iv) a third nucleic acid which codes for an evolution marker under the control of an expression control sequence which can be modulated, and where the cell is able to permit the formation of variants Y′ of the protein Y, where the variants Y′ of the protein Y are characterized by modified binding properties to the target molecule X, and
(v) where appropriate a suitable selection medium.

24. A kit for producing variants Y′ of a protein Y, comprising:
(i) a cell as defined in claim 20,
(ii) selection medium with which the cells can be selected on the basis of the modulated expression of the selection gene.
